(19) United States
(12) Patent Application Publication
(10) Pub. No.: US 2024/0148814 A1
(43) Pub. Date: May 9, 2024

(54) CYTOKINE STORM INHIBITOR

(71) Applicant: Manda Fermentation Co., Ltd., Hiroshima (JP)

(72) Inventors: SHINSUKE KISHIDA, Hiroshima (JP); KOTARO FUJIOKA, Hiroshima (JP); HIDETO TORII, Hiroshima (JP); HUANG CHUN-MING, San Diego, CA (US)

(73) Assignee: Manda Fermentation Co., Ltd., Hiroshima (JP)

(21) Appl. No.: 18/280,505

(22) PCT Filed: Mar. 11, 2022
(86) PCT No.: PCT/JP2022/010996

§ 371 Date: Sep. 6, 2023

(30) Foreign Application Priority Data
Mar. 12, 2021 (JP) 2021-040798

(51) Int. Cl. A61K 36/73; A61K 36/23; A61K 36/44; A61K 36/45; A61K 36/60; A61K 36/71; A61K 36/752; A61K 36/87; A61K 36/899; A61K 36/906

(52) U.S. Cl. CPC A61K 36/73; A61K 36/23; A61K 36/44; A61K 36/45; A61K 36/60; A61K 36/71; A61K 36/752; A61K 36/87; A61K 36/899; A61K 36/906

(57) ABSTRACT

To provide an inhibitor inhibiting a cytokine storm in a living body due to infectious disease. An inhibitor for inhibiting a cytokine storm includes a fermented composition as a main raw material, the fermented composition is obtained by fermentation and aging of a plurality of substances belonging to fruits, citrus fruits, burdock, and carrot belonging to root vegetables, cereals, sesames, seaweeds, and sugars.

### CROSS REFERENCE TO THE RELATED APPLICATIONS

This application is the national phase entry of International Application No. PCT/JP2022/010996, filed on Mar. 11, 2022, which is based upon and claims priority to Japanese Patent Application No. 2021-040798, filed on Mar. 12, 2021, the entire contents of which are incorporated herein by reference.

### TECHNICAL FIELD

The present invention relates to an inhibitor or inhibition method for inhibiting a cytokine storm, the inhibitor contains a fermented composition as a main raw material.

### BACKGROUND

Cytokines are a general term for proteinaceous factors secreted from cells involved in inflammatory and immune responses, and include several hundred or more factors. It is known that cytokines transmit signals to target cells and cause a variety of cellular responses such as cell proliferation, differentiation, cell death, and functional expression. Many molecules are related to immunity and inflammation, and various proliferation factors and proliferation inhibitory factors exist.

Cytokines individually have very diverse activity profiles and their well-balanced and cooperative interaction maintain and regulate biological functions. It is also known that cytokines play an important role as mediator substances particularly in the pathogenesis of inflammatory diseases, autoimmune diseases, and neurodegenerative diseases.

A state in which the cytokine concentration in blood is excessively increased is called “cytokine storm”. In the cytokine storm, abnormal elevation of cytokines (such as IL-1, IL-6, and TNF-α) in blood occurs due to causes such as infectious disease and drug administration, and the action thereof reaches the whole body, resulting in shock, disseminated intravascular coagulation (DIC), and multiple organ failure due to neutrophil activation, blood clotting mechanism activation, vasodilatation, and the like.

The cytokine storm is also called cytokine release syndrome (CRS) and is characterized by a poor anti-inflammatory response as well as an aggressive inflammatory response, leading to loss of homeostasis of the immune response. Important factors in the cytokine storm include TNFα, interferon, IL-1β, MCP-1 (CCL2), and IL-6.

Among them, the most important factor is IL-6.

The novel coronavirus infectious disease (COVID-19) that has caused a worldwide outbreak since the end of 2019 is an infectious disease caused by SARS-CoV-2 virus. About 80% of infected people are asymptomatic or mildly symptomatic, but about 20% of infected people have developed severe pneumonia, of which 30% have been reported to develop fatal acute respiratory distress syndrome (ARDS).

SARS-CoV-2 infects via an ACE2 receptor and activates NF-kB and SATA3 transcription factors. STAT3 enhances the production of inflammatory cytokines such as IL-6 by activating NF-kB. The cytokine storm is considered to develop acute respiratory distress syndrome (ARDS) caused by COVID-19.

Conventional techniques have disclosed several inhibitors of the cytokine storm.

### CITATION LIST

### Patent Literature

Patent Literature 1: JP 2016/104436 A1 CYTOKINE STORM INHIBITOR
Patent Literature 2: JP 2015-147778 A ANTIBODIES TO IL-6 AND USE THEREOF

### SUMMARY

### Technical Problem

However, in the conventional techniques, it has not been clearly described that increase in production of IL-6 in the process of a cytokine storm caused by the novel coronavirus (disease name: COVID-19, virus name: SARS-CoV-2) is inhibited. In the case of a medicine (including an antibody medicine) for inhibiting a cytokine storm, side effects and side reactions thereof are concerned, and new drugs are not cost-effective.

Therefore, the inventors have considered that supplements that can be ingested by a daily diet are also expected.

The present inventors found out that feeding animals with a fermented composition according to the present invention as a feed inhibits a cytokine storm caused by the novel coronavirus, thereby achieving the present invention through repeated tests and experiments, and reached the present invention.

### Solution to Problem

Technical means used by the present invention to solve the technical problems are as follows.

The present invention provides a cytokine storm inhibitor containing a fermented composition as a main raw material, the fermented composition obtained by fermentation and aging of one or more selected from apple, persimmon, banana, pineapple, Akebia, silver vine, fig, wild strawberry, strawberry, wild grape, grape, Myrica rubra, peach, plum, blueberry, and raspberry that belong to fruits, one or more selected from navel orange, Citrus hassaku, Citrus unshiu, Citrus natsudaidai, orange, i Citrus iyo, Japanese kinkan, Citrus junos, Citrus sphaerocarpa, Citrus maxima, Citrus reticulate var. poonensis, lemon, and lime that belong to citrus fruits, one or more selected from burdock, carrot, garlic, lotus root, and lily bulb that belong to root vegetables, one or more selected from brown rice, glutinous rice, white rice, millet, corn, wheat, barley, foxtail millet, and Japanese barnyard millet that belong to cereals, one or more selected from soybean, black bean, black sesame, white sesame, red bean, and walnut that belong to beans and sesames, one or more selected from sea tangle, brown seaweed, Sargassum fusiforme, green laver, and Prasiola japonica that belong to seaweeds, one or more selected from brown sugar, fructose, and glucose that belong to sugars, and one or more selected from honey, starch, cucumber, perilla, and celery, and containing the following components and amino acid compositions, each component being contained per 100 g of a main component as follows:
water: 5.0 g to 50.0 g,
protein: 0.5 g to 10.0 g,
lipid: 0.05 g to 10.00 g,
carbohydrate (saccharide): 30.0 g to 75.0 g,
carbohydrate (fiber): 0.1 g to 5.0 g,
ash: 0.5 g to 5.0 g,
β-carotene: 10 μg to 150 μg,
vitamin A potency: 10 IU to 100 IU,
vitamin B1: 0.01 mg to 0.50 mg,
vitamin B2: 0.01 mg to 0.50 mg,
vitamin B6: 0.01 mg to 0.50 mg,
vitamin E: 10.0 mg or less,
niacin: 0.1 mg to 6.0 mg,
calcium: 50 mg to 900 mg,
phosphorus: 200 mg or less,
iron: 1.0 mg to 5.0 mg,
sodium: 20 mg to 300 mg,
potassium: 300 mg to 1000 mg,
magnesium: 40 mg to 200 mg,
salt equivalent: 0.05 g to 1.00 g, and
copper: 7.0 ppm or less,
the amino acid composition being contained in 100 g as follows:
isoleucine: 30 to 200 mg,
leucine: 50 to 400 mg,
lysine: 20 to 200 mg,
methionine: 10 to 150 mg,
cystine: 10 to 100 mg,
phenylalanine: 30 to 250 mg,
tyrosine: 20 to 200 mg,
threonine: 40 to 200 mg,
tryptophan: 1 to 100 mg,
valine: 30 to 300 mg,
histidine: 10 to 200 mg,
arginine: 40 to 400 mg,
alanine: 50 to 300 mg,
aspartic acid: 100 to 600 mg,
glutamic acid: 100 to 1200 mg,
glycine: 30 to 300 mg,
proline: 40 to 400 mg, and
serine: 30 to 300 mg.

Provided is the cytokine storm inhibitor that inhibits production of IL-6 in blood and/or in a lung tissue.

The cytokine storm inhibitor may suppress production or excess production of Interleukin-6 (IL-6) caused by the novel coronavirus (disease name: COVID-19, virus name: SARS-CoV-2) or infectious disease.

Further, a cytokine storm inhibition method or an IL-6 production inhibition method in blood and/or in a lung tissue, each method using a cytokine storm inhibitor containing a fermented composition as a main raw material can be provided. The cytokine storm or elevation of IL-6 levels in blood and/or in a lung tissue can be caused by the novel coronavirus (disease name: COVID-19, virus name: SARS-CoV-2) or infectious disease.

### Advantageous Effects of Invention

The cytokine storm inhibitor of the present invention can suppress an increase or enhancement in concentration of IL-6, which is an important factor of a cytokine storm caused by the novel coronavirus (disease name: COVID-19, virus name: SARS-CoV-2). The cytokine storm inhibitor can be used as a prophylactic or therapeutic agent for exacerbation of COVID-19 infectious disease.

Hereinafter, the present invention will be further described by using the accompanying drawings and Examples.

### BRIEF DESCRIPTION OF THE DRAWINGS

FIG. 1 shows reproduction test data of a cytokine storm caused by the novel coronavirus-related proteins.

FIG. 2 shows data of a concentration dependency caused by the novel coronavirus-related proteins.

FIG. 3 shows data of an inhibitory effect caused by the novel coronavirus-related proteins.

### DETAILED DESCRIPTION OF THE EMBODIMENTS

The inventor of the present invention focused on the present fermented composition and tested whether it affects a cytokine storm caused by the infection of the novel corona virus (disease name: COVID-19, virus name: SARS-CoV-2).

First, they inoculated with 10 μg/50 μl of a phosphate buffer solution (PBS) containing proteins that is a part of the novel coronavirus (disease name: COVID-19, virus name: SARS-CoV-2) into the nasal mucosa the mice, or a PBS solution of a fluorescent protein as a negative. Green Fluorescent Protein (GFP) was used as a fluorescent protein. The protein, which is a part of the novel coronavirus (disease name: COVID-19, virus name: SARS-CoV-2), is a spike protein (YP_009724390.1) of (SARS-CoV-2), which is s1 (including a receptor binding domain (RBD)) and S2 ranging from amino acid residues 14 to 1209, and is produced and adjusted by expression in HEK293 cells.

Mice inoculated with solely a phosphate buffer solution (PBS) were also prepared to obtain standard data which will serve as a basis.

These three types of mice were left held in a cage for 6 hours. Thereafter, these mice were dissected and the amount of IL-6 in the lungs was measured by ELISA. Then, a numerical value was obtained by subtracting a numerical value (baseline) indicating IL-6 in the group of the mice inoculated with solely PBS from a numerical value in the group inoculated with a coronavirus-related protein.

The numerical value of in the group inoculated with solely PBS was subtracted for the purpose of omitting the influence by PBS because the coronavirus protein is mixed with the PBS solution before it is inoculated into the nasal mucosa of the mice. Similarly, the influence of the PBS solution was eliminated in the group inoculated with the fluorescent protein.

The comparison with fluorescent protein inoculation was simply intended to show that the protein component did not affect the increase or decrease in IL-6 levels.

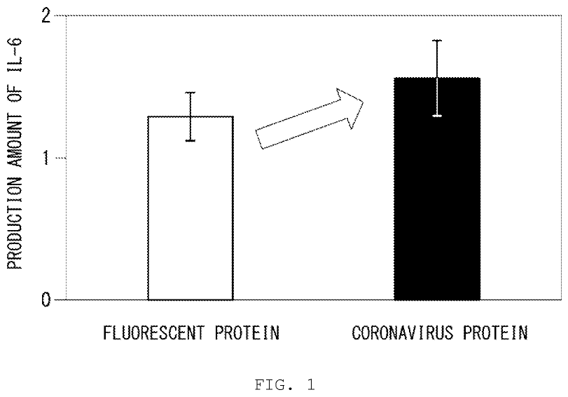

FIG. 1 shows reproduction test data of a cytokine storm caused by a novel coronavirus-related protein. This result showed that an increase in production amount of IL-6 was observed in the protein, which is a part of the novel coronavirus, that is, the cytokine storm could be reproduced in mice.

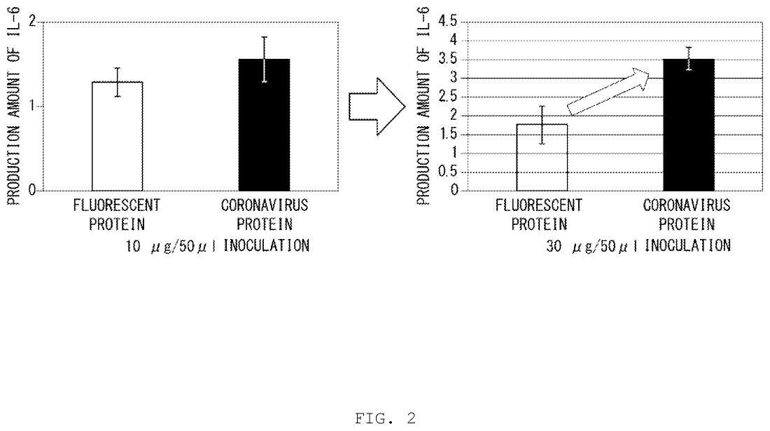

FIG. 2 shows data indicating concentration dependency of a cytokine storm caused by a novel coronavirus-related protein. The data calculation method is the same as in the reproduction test data on a cytokine storm caused by a novel coronavirus-related protein.

The nasal mucosa of each mouse was inoculated with a fluorescent protein or a protein that compose a part of the novel coronavirus, in amounts of 10 μg and 30 μg, respectively. From the results in FIG. 2, in the case of 10 μg and 30 μg of the fluorescent protein, concentration dependency cannot be confirmed. On the other hand, it was shown that when the protein, which is a part of the novel coronavirus, was increased in amounts of 10 μg and 30 μg, respectively, the production amount of IL-6 increased about 2 times in a concentration-dependent manner.

The present results show that increasing the amount of the protein, which is a part of the novel coronavirus, to be inoculated into the nasal mucosa of each mouse, increases the production amount of IL-6 in a concentration-dependent manner, which supports correct operation of the present test system.

As the present test system was established, a test of how the fermented composition of the present invention affects the cytokine storm caused by infectious disease was conducted.

In this test, following fermented composition of the present invention was used. That is, the fermented composition is obtained by fermentation and aging of one or more selected from apple, persimmon, banana, pineapple, Akebia, silver vine, fig, wild strawberry, strawberry, wild grape, grape, Myrica rubra, peach, plum, blueberry, and raspberry that belong to fruits, one or more selected from navel orange, Citrus hassaku, Citrus unshiu, Citrus natsudaidai, orange, Citrus iyo, Japanese kinkan, Citrus junos, Citrus sphaerocarpa, Citrus maxima, Citrus reticulate var. poonensis, lemon, and lime that belong to citrus fruits, one or more selected from burdock, carrot, garlic, lotus root, and lily bulb that belong to root vegetables, one or more selected from brown rice, glutinous rice, white rice, millet, corn, wheat, barley, foxtail millet, and Japanese barnyard millet that belong to cereals, one or more selected from soybean, black bean, black sesame, white sesame, red bean, and walnut that belong to beans and sesames, one or more selected from sea tangle, brown seaweed, Sargassum fusiforme, green laver, and Prasiola japonica that belong to seaweeds, one or more selected from brown sugar, fructose, and glucose that belong to sugars, and one or more selected from honey, starch, cucumber, perilla, and celery, and contains the following components and amino acid compositions, each component being contained per 100 g of a main component as follows: water: 5.0 g to 50.0 g, protein: 0.5 g to 10.0 g, lipid: 0.05 g to 10.00 g, carbohydrate (saccharide): 30.0 g to 75.0 g, carbohydrate (fiber): 0.1 g to 5.0 g, ash: 0.5 g to 5.0 g, β-carotene: 10 μg to 150 μg, vitamin A potency: 10 IU to 100 IU, vitamin B1: 0.01 mg to 0.50 mg, vitamin B2: 0.01 mg to 0.50 mg, vitamin B6: 0.01 mg to 0.50 mg, vitamin E: 10.0 mg or less, niacin: 0.1 mg to 6.0 mg, calcium: 50 mg to 900 mg, phosphorus: 200 mg or less, iron: 1.0 mg to 5.0 mg, sodium: 20 mg to 300 mg, potassium: 300 mg to 1000 mg, magnesium: 40 mg to 200 mg, salt equivalent: 0.05 g to 1.00 g, and copper: 7.0 ppm or less, the amino acid composition being contained in 100 g as follows: isoleucine: 30 to 200 mg, leucine: 50 to 400 mg, lysine: 20 to 200 mg, methionine: 10 to 150 mg, cystine: 10 to 100 mg, phenylalanine: 30 to 250 mg, tyrosine: 20 to 200 mg, threonine: 40 to 200 mg, tryptophan: 1 to 100 mg, valine: 30 to 300 mg, histidine: 10 to 200 mg, arginine: 40 to 400 mg, alanine: 50 to 300 mg, aspartic acid: 100 to 600 mg, glutamic acid: 100 to 1200 mg, glycine: 30 to 300 mg, proline: 40 to 400 mg, and serine: 30 to 300 mg, and the fermented composition is used as a main raw material, fermented and aged for 3 years or more under temperature control, and subjected to trituration, inspection, and packing to obtain a product.

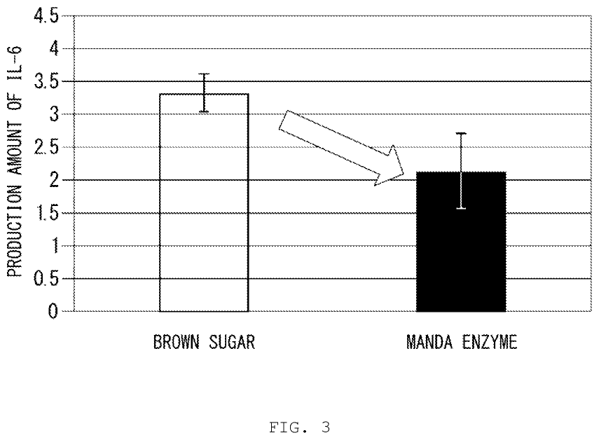

FIG. 3 shows an inhibitory effect of the present fermented composition on a cytokine storm caused by a novel coronavirus-related protein.

Mice are fed 2% brown sugar (control group) or 200 ul of Manda Enzyme (the fermented composition of the present invention) for 30 days without letting them develop diarrhea. The nasal mucosa of each mouse in the experimental group is then inoculated with 30 μg/50 μl of a protein, which is a part of the novel coronavirus. On the other hand, the nasal mucosa of each mouse in the control group is inoculated with 50 μl of PBS. 30 μg/50 μl of the protein, which is a part of the novel coronavirus, was mixed in the PBS solution, and it was compared with the group inoculated with only the PBS solution as a control group.

After the nasal mucosa of each mouse was inoculated with the protein, which is a part of the novel coronavirus, or the PBS solution, these mice were left for 6 hours and then dissected to extract the lungs. The amount of IL-6 in each lung tissue was measured by ELISA.

FIG. 3 shows data that represents an inhibitory effect of the novel coronavirus-related protein on a cytokine storm of the present fermented composition. In the brown sugar group (control group), the graph shows the difference in the amount of IL-6, in mice inoculated with some proteins that make up the new coronavirus to those given brown sugar, and IL-6 in mice given only PBS solution.

Similarly, in the Manda Enzyme (fermented composition of the present invention) group, the graph shows the difference in IL-6 levels between mice fed with Manda Enzyme and those inoculated with some proteins that make up the new coronavirus, and those given only PBS solution. In the Manda Enzyme feeding group, it has shown that the amount of IL-6 production was suppressed compared to those fed with the brown sugar group. Therefore, it has become clear that ingestion of Manda Enzyme (the fermented composition of the present invention) has an inhibitory effect on a cytokine storm in a mouse system.

What is claimed is:

1. A cytokine storm inhibitor comprising a fermented composition as a main raw material, wherein the fermented composition is obtained by fermentation and aging of
at least one selected from the group consisting of apple, persimmon, banana, pineapple, Akebia, silver vine, fig, wild strawberry, strawberry, wild grape, grape, Myrica rubra, peach, plum, blueberry, and raspberry that belong to fruits,
at least one selected from the group consisting of navel orange, Citrus hassaku, Citrus unshiu, Citrus natsudaidai, orange, Citrus iyo, Japanese kinkan, Citrus junos, Citrus sphaerocarpa, Citrus maxima, Citrus reticulate var. poonensis, lemon, and lime that belong to citrus fruits,
at least one selected from the group consisting of burdock, carrot, garlic, lotus root, and lily bulb that belong to root vegetables,
at least one selected from the group consisting of brown rice, glutinous rice, white rice, millet, corn, wheat, barley, foxtail millet, and Japanese barnyard millet that belong to cereals,
at least one selected from the group consisting of soybean, black bean, black sesame, white sesame, red bean, and walnut that belong to beans and sesames,
at least one selected from the group consisting of sea tangle, brown seaweed, Sargassum fusiforme, green laver, and Prasiola japonica that belong to seaweeds,
at least one selected from the group consisting of brown sugar, fructose, and glucose that belong to sugars, and
at least one selected from the group consisting of honey, starch, cucumber, perilla, and celery, and
containing the following components and amino acid compositions, each component being contained per 100 g of a main component as follows:
water: 5.0 g to 50.0 g,
protein: 0.5 g to 10.0 g,
lipid: 0.05 g to 10.00 g,
carbohydrate (saccharide): 30.0 g to 75.0 g,
carbohydrate (fiber): 0.1 g to 5.0 g,
ash: 0.5 g to 5.0 g,
β-carotene: 10 μg to 150 μg,
vitamin A potency: 10 IU to 100 IU,
vitamin B1: 0.01 mg to 0.50 mg,
vitamin B2: 0.01 mg to 0.50 mg,
vitamin B6: 0.01 mg to 0.50 mg,
vitamin E: 10.0 mg or less,
niacin: 0.1 mg to 6.0 mg,
calcium: 50 mg to 900 mg,
phosphorus: 200 mg or less,
iron: 1.0 mg to 5.0 mg,
sodium: 20 mg to 300 mg,
potassium: 300 mg to 1000 mg,
magnesium: 40 mg to 200 mg,
salt equivalent: 0.05 g to 1.00 g, and
copper: 7.0 ppm or less,
the amino acid composition being contained in 100 g as follows:
isoleucine: 30 to 200 mg,
leucine: 50 to 400 mg,
lysine: 20 to 200 mg,
methionine: 10 to 150 mg,
cystine: 10 to 100 mg,
phenylalanine: 30 to 250 mg,
tyrosine: 20 to 200 mg,
threonine: 40 to 200 mg,
tryptophan: 1 to 100 mg,
valine: 30 to 300 mg,
histidine: 10 to 200 mg,
arginine: 40 to 400 mg,
alanine: 50 to 300 mg,
aspartic acid: 100 to 600 mg,
glutamic acid: 100 to 1200 mg,
glycine: 30 to 300 mg,
proline: 40 to 400 mg, and
serine: 30 to 300 mg.

2. The cytokine storm inhibitor according to claim 1, wherein the cytokine storm inhibitor inhibits a production of IL-6 in blood and/or in a lung tissue.

3. The cytokine storm inhibitor according to claim 1, wherein a production of IL-6 caused by novel coronavirus or infectious disease is inhibited.

4. A cytokine storm inhibition method using a cytokine storm inhibitor containing a fermented composition as a main raw material, wherein the fermented composition is obtained by fermentation and aging of
at least one selected from the group consisting of apple, persimmon, banana, pineapple, Akebia, silver vine, fig, wild strawberry, strawberry, wild grape, grape, Myrica rubra, peach, plum, blueberry, and raspberry that belong to fruits,
at least one selected from the group consisting of navel orange, Citrus hassaku, Citrus unshiu, Citrus natsudaidai, orange, Citrus iyo, Japanese kinkan, Citrus junos, Citrus sphaerocarpa, Citrus maxima, Citrus reticulate var. poonensis, lemon, and lime that belong to citrus fruits,
at least one selected from the group consisting of burdock, carrot, garlic, lotus root, and lily bulb that belong to root vegetables,
at least one selected from the group consisting of brown rice, glutinous rice, white rice, millet, corn, wheat, barley, foxtail millet, and Japanese barnyard millet that belong to cereals,
at least one selected from the group consisting of soybean, black bean, black sesame, white sesame, red bean, and walnut that belong to beans and sesames,
at least one selected from the group consisting of sea tangle, brown seaweed, Sargassum fusiforme, green laver, and Prasiola japonica that belong to seaweeds,
at least one selected from the group consisting of brown sugar, fructose, and glucose that belong to sugars, and
at least one selected from the group consisting of honey, starch, cucumber, perilla, and celery, and
containing the following components and amino acid compositions, each component being contained per 100 g of a main component as follows:
water: 5.0 g to 50.0 g,
protein: 0.5 g to 10.0 g,
lipid: 0.05 g to 10.00 g,
carbohydrate (saccharide): 30.0 g to 75.0 g,
carbohydrate (fiber): 0.1 g to 5.0 g,
ash: 0.5 g to 5.0 g,
β-carotene: 10 μg to 150 μg,
vitamin A potency: 10 IU to 100 IU,
vitamin B1: 0.01 mg to 0.50 mg,
vitamin B2: 0.01 mg to 0.50 mg,
vitamin B6: 0.01 mg to 0.50 mg,
vitamin E: 10.0 mg or less,
niacin: 0.1 mg to 6.0 mg,
calcium: 50 mg to 900 mg,
phosphorus: 200 mg or less,
iron: 1.0 mg to 5.0 mg,
sodium: 20 mg to 300 mg,
potassium: 300 mg to 1000 mg,
magnesium: 40 mg to 200 mg,
salt equivalent: 0.05 g to 1.00 g, and
copper: 7.0 ppm or less,
the amino acid composition being contained in 100 g as follows:
isoleucine: 30 to 200 mg,
leucine: 50 to 400 mg,
lysine: 20 to 200 mg,
methionine: 10 to 150 mg,
cystine: 10 to 100 mg,
phenylalanine: 30 to 250 mg,
tyrosine: 20 to 200 mg,
threonine: 40 to 200 mg,
tryptophan: 1 to 100 mg,
valine: 30 to 300 mg,
histidine: 10 to 200 mg,
arginine: 40 to 400 mg,
alanine: 50 to 300 mg,
aspartic acid: 100 to 600 mg,
glutamic acid: 100 to 1200 mg,
glycine: 30 to 300 mg,
proline: 40 to 400 mg, and
serine: 30 to 300 mg.
